(19) United States
(12) Patent Application Publication
(10) Pub. No.: US 2019/0139631 A1
(43) Pub. Date: May 9, 2019

(54) ESTIMATION AND USE OF CLINICIAN ASSESSMENT OF PATIENT ACUITY

(71) Applicant: KONINKLIJKE PHILIPS N.V., EINDHOVEN (NL)

(72) Inventors: Larry James ESHELMAN, OSSINING, NY (US); Eric Thomas CARLSON, NEW YORK, NY (US); Lin YANG, CHANDLER, AZ (US); Minnan XU, CAMBRIDGE, MA (US); Bryan CONROY, GARDEN CITY SOUTH, NY (US)

(21) Appl. No.: 16/097,299

(22) PCT Filed: May 4, 2017
(86) PCT No.: PCT/EP2017/060591

§ 371 Date: Oct. 29, 2018

Related U.S. Application Data
(60) Provisional application No. 62/331,496, filed on May 4, 2016.

(51) Int. Cl. G16H 10/60; G16H 50/20; G06N 20/00

(52) U.S. Cl. CPC G16H 10/60; G06N 20/00; G16H 50/20

(57) ABSTRACT

The present disclosure relates to estimation and use of clinician assessment of patient acuity. In various embodiments, a plurality of patient feature vectors associated with a plurality of respective patients may be obtained (302, 304). Each patient feature vector may include one or more health indicator features indicative of observable health indicators of a patient, and one or more treatment features indicative of characteristics of treatment provided to the patient. A machine learning model (216) may be trained (306) based on the patient feature vectors to receive, as input, subsequent patient feature vectors, and to provide, as output, indications of levels of clinician acuity assessment. Later, a patient feature vector associated with a given patient may be provided (404) as input to the machine learning model. Based on output from the machine learning model, a level of clinician acuity assessment associated with the given patient may be estimated (406) and used (408-416) for various applications.

### TECHNICAL FIELD

Various embodiments described herein are directed generally to health care. More particularly, but not exclusively, various methods and apparatus disclosed herein relate to estimation and use of clinician assessment of patient acuity.

### BACKGROUND

Various techniques exist for assessing deterioration of, and/or medical care required by, a patient (i.e. “patient acuity”) based on a variety of health indicators. These health indicators may include but are not limited to age, gender, weight, height, blood pressure, lactose levels, blood sugar, temperature, genetic history, and so forth. Clinical decision support (CDS) algorithms may use these health indicators to provide an assessment of the patient acuity. Generally, CDS algorithms are used as a supplement to the decision-making of the health professional, rather than a replacement therefor.

While CDS algorithms can oftentimes alert a clinician to the existence of previously unknown changes in patient condition, in other circumstances, the clinician may already be aware of the change (e.g., deterioration in acuity). In such a case, the CDS algorithm does not offer new information to the clinician and, instead, may serve as little more than an annoyance. If this scenario occurs repeatedly, the clinician may begin to ignore the output of the CDS algorithm altogether.

### SUMMARY

The present disclosure is directed to inventive methods and apparatus for estimating and utilizing clinician assessment of patient acuity. In various embodiments, historical data pertaining to health indicators associated with a plurality of patients, as well as characteristics of treatments provided to those patients, may be used to establish a methodology for estimating a clinician acuity assessment index (“CAM”). In some implementations, establishing such a methodology may include training a machine learning model. An estimated CAAI may then be used for various purposes.

In some embodiments, the CAAI may be used in conjunction with another indicator of patient acuity, e.g., to determine whether a current clinician assessment of the patient's acuity is accurate. In some embodiments, the CAM may be taken into account when making a variety of medical decisions, such as determining whether to admit-discharge-transfer (“ADT”) patients, institute various treatments or surgeries, alter medical alarms associated with patients, and so forth. In some embodiments, the CAAI may be used as a more robust and/or accurate indicator of patient acuity than another indicator which takes into account only health indicators.

Additionally or alternatively, the CAAI may be communicated (e.g., as output on a computing device) to various medical personnel for various purposes. For example, the CAAI may be provided to a doctor just starting her shift who may not otherwise have immediate knowledgeable of the patient's acuity, so that the doctor can more quickly become up to speed. As another example, the CAAI may be provided to nurses to guide how closely the nurses should monitor the patient. As yet another example, the CAAI may be provided to medical technicians to guide how the technicians tune or otherwise configure medical equipment.

Examples described throughout this disclosure are implemented using a machine learning classifier. However, this is not meant to be limiting. Generally speaking, techniques described herein may be performed in other ways as well. For example, in some implementations, a CAAI for a patient-of-interest may be determined using one or rules (e.g., heuristics) established as part of hospital procedures and policies. That CAAI may then be used for various purposes as described above, with or without the use of computers.

Generally, in one aspect, a plurality of patient feature vectors associated with a plurality of respective patients may be obtained. Each patient feature vector may include one or more health indicator features indicative of one or more observable health indicators of a patient, and one or more treatment features indicative of one or more characteristics of treatment provided to the patient. A machine learning classifier may be trained based on the patient feature vectors to receive, as input, subsequent patient feature vectors, and to provide, as output, indications of levels of clinician acuity assessment. Later, a patient feature vector associated with a given patient may be obtained and provided as input to the machine learning classifier. Based on output from the machine learning classifier, a level of clinician acuity assessment associated with the given patient may be estimated.

In various embodiments, the estimated level of clinician acuity assessment of the given patient may be determined to fail to satisfy a clinician acuity assessment threshold. Consequently, output may be provided to medical personnel to instruct the medical personnel that a current clinician assessment of the given patient's acuity is inaccurate.

In various embodiments, it may be determined that an objective acuity level of the given patient does not match the level of clinician acuity assessment of the given patient. In various versions, output may be provided to medical personnel to instruct the medical personnel that a current clinician assessment of the patient's acuity is inaccurate. In various versions, an alteration may be made to a manner in which an indicator of an objective acuity level of the given patient is output to medical personnel to notify the medical personnel that additional concern for the given patient is warranted.

In various embodiments, at least one patient feature vector includes a feature indicative of whether a health parameter of a patient is being measured invasively or non-invasively. In various embodiments, at least one patient feature vector includes a feature indicative of a frequency at which a health indicator of a patient is measured. In various embodiments, at least one patient feature vector includes a feature indicative of whether a patient is supported by a life-critical system. In various embodiments, at least one patient feature vector includes a feature indicative of a dosage or duration of a medication administered to a patient. In various embodiments, each of the plurality of patient feature vectors includes a label indicative of an outcome associated with the respective patient.

As used herein, “patient acuity” is used to refer to a measure of medical care required and/or warranted by a patient. It may also refer to a closely related concept of patient deterioration, which correlates a level of a patient's deterioration (e.g., how rapidly) to an amount of medical care warranted by the patient. For example, a severely injured patient experiencing hemorrhaging and/or other life-threatening symptoms may require intensive medical care, and thus may have a higher patient acuity than, say, a stabilized patient for which the best treatment is time and rest. “Medical personnel,” or “clinicians” as used herein, may include but are not limited to doctors, nurses, nurse practitioners, therapists, technicians, and so forth.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Various embodiments described herein relate to a system including: one or more processors; and memory coupled with the one or more processors, the memory storing instructions that, in response to execution of the instructions by the one or more processors, cause the one or more processors to: obtain a plurality of patient feature vectors associated with a plurality of patients, each patient feature vector including a plurality of health indicator features associated with a patient of the plurality of patients, and a plurality of treatment features associated with treatment of the patient by medical personnel based at least in part on the plurality of health indicator features associated with the patient; and train a machine learning model based on the patient feature vectors to receive, as input, subsequent patient feature vectors, and to provide, as output, indications of levels of clinician acuity assessment.

Various embodiments described herein relate to a computer-implemented method, including: obtaining, by one or more processors, a patient feature vector associated with a given patient, the patient feature vector including one or more health indicator features indicative of one or more observable health indicators of the given patient, and one or more treatment features indicative of one or more characteristics of treatment provided to the given patient; providing, by the one or more processors, as input to a machine learning model operated by the one or more processors, the patient feature vector; and estimating, by the one or more processors, based on output from the machine learning model, a level of clinician acuity assessment associated with the given patient.

Various embodiments described herein relate to a non-transitory computer-readable medium including instructions that, in response to execution of the instructions by a computing system, cause the computing system to perform the following operations: obtaining a plurality of patient feature vectors associated with a plurality of respective patients, each patient feature vector including one or more health indicator features indicative of one or more observable health indicators of a patient, and one or more treatment features indicative of one or more characteristics of treatment provided to the patient; training a machine learning model based on the patient feature vectors to receive, as input, subsequent patient feature vectors, and to provide, as output, indications of levels of clinician acuity assessment; obtaining a patient feature vector associated with a given patient; providing, as input to the machine learning model, the patient feature vector; and estimating, based on output from the machine learning model, a level of clinician acuity assessment associated with the given patient.

By establishing a machine learning model to estimate what the clinician already understands about a patient condition (i.e., the clinician acuity assessment), the system may be able to more intelligently select how to present “objective” acuity assessments (e.g., outputs of CDS algorithms) to the clinician and other staff. Where the clinician acuity assessment already matches the objective acuity assessment, a conclusion can be drawn that the clinician is already aware of the condition and alarms (or other active notifications) can be suppressed in favor of more passive notification (or even no notification) to reduce the likelihood that the clinician will begin to view the objective acuity assessment as useless or otherwise begin to ignore it (e.g., due to alarm fatigue). Conversely, more active notification measures may then be reserved for the case where there is a discrepancy between the clinician and objective acuity assessments, where it is more likely that the objective acuity assessment will provide the clinician with new information.

Various embodiments are described wherein the memory further includes instructions to: provide one or more feature vectors that include health indicator features and treatment features associated with a given patient to the machine learning model as input; and estimate a level of clinician acuity assessment of the given patient based on output of the machine learning model.

Various embodiments additionally include instructions to determine that the estimated level of clinician acuity assessment of the given patient fails to satisfy a clinician acuity assessment threshold; and cause output to be provided to medical personnel to instruct the medical personnel that a current clinician assessment of the given patient's acuity is inaccurate.

Various embodiments additionally include instructions to determine that an objective acuity level of the given patient does not match the level of clinician acuity assessment of the given patient.

Various embodiments additionally include instructions to cause output to be provided to medical personnel to instruct the medical personnel that a current clinician assessment of the patient's acuity is inaccurate.

Various embodiments additionally include instructions to alter a manner in which an indicator of an objective acuity level of the given patient is output to medical personnel to notify the medical personnel that additional concern for the given patient is warranted.

Various embodiments are described wherein at least one patient feature vector includes a feature indicative of whether a health parameter of a patient is being measured invasively or non-invasively.

Various embodiments are described wherein at least one patient feature vector includes a feature indicative of a frequency at which a health indicator of a patient is measured.

Various embodiments are described wherein at least one patient feature vector includes a feature indicative of whether a patient is supported by a life-critical system.

Various embodiments are described wherein at least one patient feature vector includes a feature indicative of whether a patient is supported by a life-critical system.

Various embodiments are described wherein each of the plurality of patient feature vectors includes a label indicative of an outcome associated with the respective patient.

Some implementations are directed to utilization of the trained model. For example, the trained model may be utilized in the iterative updating and further developing the trained model and updating the same. Such could be accomplished in various embodiments by entering the various patient feature vectors into the previously trained models, the patient feature vectors provided as input to the already trained model. In uses, patient feature vectors associated with given patients may be obtained and provided as input to the machine learning model. In use and after entry of the patient feature vectors, the output of the machine learning model may include an estimated level of clinician acuity assessment associated with the given patient and the patient feature vector. Thus, in various examples, a method of using a trained machine learning model to generate a CAM, obtain an objective measure, compare and select alarm characteristics may also be provided.

In some implementations, a method is provided that includes generating a candidate CAAI resulting from patient feature vectors is provided. The method further includes entering the current patient feature vectors and treatment vectors as input to a trained machine learning classifier and generating, over the trained model, an estimated level of clinician acuity assessment as output for the associated patient. As well, the estimated level of clinician acuity assessment may be generated through using the trained machine learning model set forth.

In some aspects, a computer implemented method of using a trained machine learning model is described wherein the method includes obtaining, by one or more processors, a patient feature vector and a treatment feature vector, both associated with a given patient; providing, by the one or more processors, as input to a machine learning model operated by the one or more processors, the patient feature vector and the treatment feature vector; and estimating, by the one or more processors, based on output from the machine learning model, a level of clinician acuity assessment associated with the given patient. Further, in various implementations use of a trained machine learning model is described wherein the machine learning model is trained using the various computer implemented training method steps described herein.

In some implementations, the training of the machine learning model comprises performing backpropagation on the convolutional network based on the training output of the plurality of training examples.

Other implementations may include a non-transitory computer readable storage medium storing instructions executable by a processor (e.g., a central processing unit (CPU) to perform a method such as one or more of the methods described above. Yet another implementation may include a system of one or more computers and/or one or more learning models that include one or more processors operable to execute stored instructions to perform a method such as one or more of the methods described above.

Various embodiments relate to a method for presenting clinical decision support information to a clinician, a device for performing the method, and a non-transitory machine-readable storage medium encoded with instructions for executing the method, the method including: receiving a plurality of features descriptive of a patient; applying a first trained model to at least a first portion of the plurality of features to generate a patient acuity value as an estimate of a patient condition; applying a second trained model to at least a second portion of the plurality of features to generate a clinician acuity assessment value as an estimate of a clinician's assessment of the patient condition; comparing the patient acuity value to the clinician acuity assessment value; and determining at least one presentation characteristic for presenting the patient acuity value based on the comparison of the patient acuity value to the clinician acuity assessment value.

Various embodiments are described wherein the second portion of the plurality of features includes at least one characteristic of a treatment provided to the patient.

Various embodiments additionally include suppressing an alarm generated based on the patient acuity value when the comparison of the patient acuity value to the clinician acuity assessment value determines that the clinician acuity assessment value is substantially the same as the patient acuity value.

Various embodiments are described wherein the step of determining comprises: selecting attention-drawing presentation characteristics when the comparison of the patient acuity value to the clinician acuity assessment value determines that the clinician acuity assessment value is substantially different from the patient acuity value. As will be understood, attention-drawing presentation characteristics may include various characteristics that are capable of capturing a clinician's attention when the clinician is not viewing or only casually glancing at an output monitor. For example, increasing a text size for the output patient acuity value, changing the color of the patient acuity value to stand out with relation to the other information output on the screen, causing the patient acuity value to blink, or outputting an audible sound to draw attention. In some embodiments, the attention-drawing presentation characteristics may be a predefined set of one or more characteristics selected to be “attention drawing” that is used when (in some embodiments, only when) the clinician acuity assessment value does not substantially match the patient acuity value. Various embodiments are described wherein the at least one presentation characteristic includes at least one of: an audible sound, text size, text color, and a text blink setting.

It should be appreciated that all combinations of the foregoing concepts and additional concepts described in greater detail herein are contemplated as being part of the subject matter disclosed herein. For example, all combinations of claimed subject matter appearing at the end of this disclosure are contemplated as being part of the subject matter disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating various principles of the embodiments described herein.

FIG. 1A demonstrates how a conventional patient acuity index may be determined based on a plurality of health indicators.

FIG. 1B demonstrates how a clinician acuity assessment index may be determined using techniques disclosed herein based on a plurality of health indicators and treatment characteristics, in accordance with various embodiments.

FIG. 2 schematically illustrates an environment in which disclosed techniques may be employed, in accordance with various embodiments.

FIG. 3 schematically illustrates an example method of training a machine learning classifier configured with selected aspects of the present disclosure, in accordance with various embodiments.

FIG. 4 schematically illustrates an example method of estimating CAM and using that estimate for various purposes, in accordance with various embodiments.

FIG. 5 schematically depicts components of an example computer system, in accordance with various embodiments.

### DETAILED DESCRIPTION

Various techniques exist for assessing patient acuity based on a variety of health indicators. However, observed health indicators may not necessarily provide a comprehensive view of patient acuity. Medical treatment provided by medical personnel to patients may itself also be highly indicative of patient acuity. Thus, there is a need in the art to take into account characteristics of treatment provided by clinicians to estimate clinician assessment of patient acuity, and to utilize the ascertained clinician assessment of patient acuity in various ways. More generally, Applicants have recognized and appreciated that it would be beneficial to predict and/or estimate a clinician acuity assessment of a patient based on a variety of signals, such as medical indicators and/or characteristics of treatment provided to the patient. By taking into account the clinician acuity assessment (i.e., an estimate of how the clinician currently views the patient's state), the system can more intelligently determine how to output the output of a related patient acuity measure. For example, if the clinician acuity assessment for acute kidney injury (AKI) roughly matches the “conventional” assessment of AKI by another CDS algorithm, the output of the objective assessment may be presented in a passive manner (e.g., simply displayed on a screen of a monitor) whereas if the clinician's acuity assessment for the AKI is much lower (i.e., less sever in this example) than the objective AKI CDS algorithm, the output may be more actively presented (e.g., flashing text, alarms, messages sent to attending clinicians, etc.). In view of the foregoing, various embodiments and implementations of the present invention are directed to estimating and utilizing clinician assessment of patient acuity.

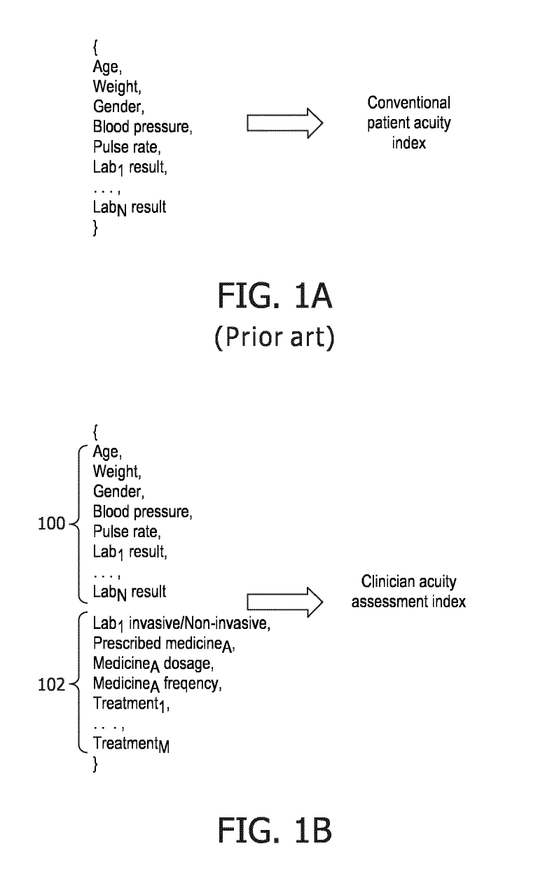

Referring to FIG. 1A, an example of how a “conventional” patient acuity index may be determined is shown. A variety of so-called “health indicators” (e.g., observable attributes) associated with a patient may be used to determine the patient's acuity. In this example, the patient's age, weight, gender, blood pressure, pulse rate, and results from a plurality of labs LAB_{1-N} are used to determine an acuity index (or “score”) associated with the patient. Other health indicators such as temperature, glucose levels, oxygen levels, etc., may be used in addition to or instead of those depicted in FIG. 1A. While such a traditional index may be useful in assessing acuity of the patient, it fails to account for clinician expertise and/or experience in diagnosing and/or treating various ailments and disorders. In some cases, the traditional index may simply reflect what the clinician already knows and, as such, may constitute redundant information.

Accordingly, in various embodiments, techniques described herein may determine a so-called “clinician acuity assessment index”, or “CAAI”, for a patient. In addition to taking into account one or more health indicators shown in FIG. 1A, the CAM may take into account one or more characteristics of treatment provided to the patient by medical personnel. In many instances, characteristics of treatment provided to a patient may more strongly reflect clinician concern for the patient (and hence, patient acuity) than the objective health indicators themselves. As will be described herein, the CAAI may be used for a variety of purposes.

FIG. 1B depicts an example of how disclosed techniques may be used to determine a CAAI, in accordance with various embodiments. As indicated generally at 100, one or more of the same health indicators that were taken into account in FIG. 1A may be taken into account. However, as indicated generally at 102, one or more characteristics of treatment provided to the patient may also be taken into account, in addition to or instead of the health indicators. In this example, the treatment characteristics that are taken into account to determine the CAAI include a manner in which a particular lab (LAB₁) was performed (invasive or non-invasive), a prescribed (or administered) medicine, MEDICINE_{A}, a dosage of MEDICINE_{A} prescribed (and/or administered), a frequency at which MEDICINE_{A} is administered (and/or prescribed to be administered), and a plurality of other treatment characteristics (labeled TREATMENT₁ . . . TREATMENT_{M} in FIG. 1B). These are just examples of treatment characteristics that may be taken into account, and are not meant to be limiting. The CAM estimated using these features may in many cases be more robust and/or more accurately reflect patient acuity than other conventional indices.

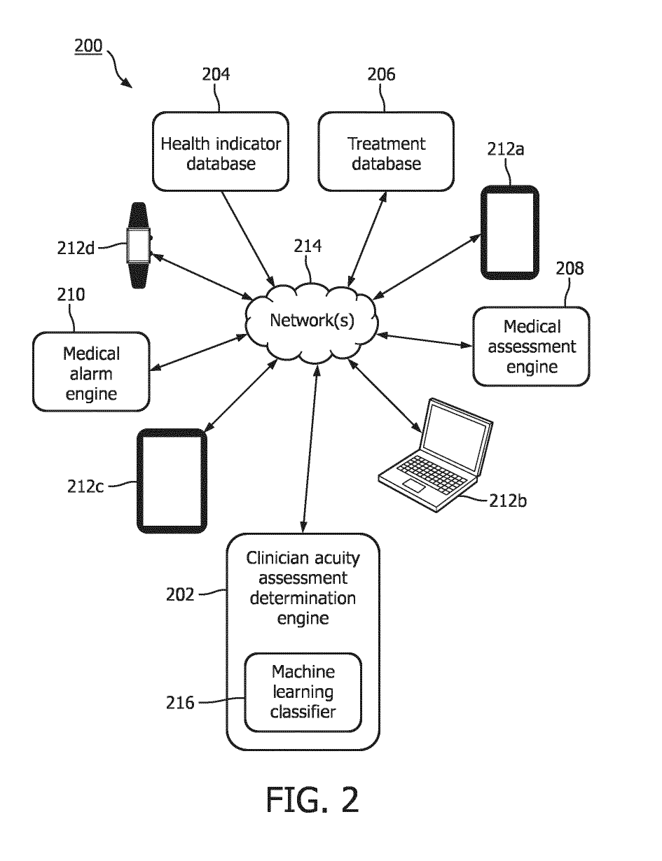

FIG. 2 depicts an example environment 200 in which various components may interoperate to perform techniques described herein. The environment 200 includes a variety of components that may be configured with selected aspects of the present disclosure, including a clinician assessment determination engine 202, one or more health indicator databases 204, one or more treatment databases 206, one or more medical assessment engines 208, and/or one or more medical alarm engines 210. A variety of client devices 212, such as a smart phone 212a, a laptop computer 212b, a tablet computer 212c, and a smart watch 212d, may also be in communication with other components depicted in FIG. 2. In some embodiments, the components of FIG. 2 may be communicatively coupled via one or more wireless or wired networks 214, although this is not required. And while the components are depicted in FIG. 2 separately, it should be understood that one or more components depicted in FIG. 2 may be combined in a single computer system (which may include one or more processors), and/or implemented across multiple computer systems (e.g., across multiple servers).

Clinician assessment determination engine 202 may be configured to determine a CAAI for one or more patients based on a variety of treatment characteristics. In some embodiments, clinician assessment determination engine 202 may include one or more machine learning classifiers 216 that may be trained to receive, as input pertaining to a patient, one or more feature vectors containing health indicator and treatment features, and to provide, as output, CAAIs estimated based on the input. The output of machine learning classifier 216 may be used by various components described herein in various ways. While various embodiments are described herein with respect to use of machine learning classifiers to create CAAIs as well as objective patient acuity indicators, it will be apparent that various embodiments may additionally or alternatively use other machine learning models such as, for example, linear regression models which may be useful where the acuity indices are to be represented as numerical values.

Health indicator database 204 may include records of observed and/or observable health indicators associated with a plurality of patients. For example, health indicator database 204 may include a plurality of patient records that include, among other things, data indicative of one or more health indicators of the patients. Example health indicators are described elsewhere herein. In other embodiments, health indicator database may include anonymized health indicators associated with a plurality of patients, e.g., collected as part of a study.

Treatment database 206 may include information pertaining to treatment of patients by medical personnel, include various characteristics of treatment provided to patients that might not otherwise be contained in health indicator database 204. For example, whereas health indicator database 204 may include various vital sign measurements of a plurality of patients, such as blood pressure, pulse rate, blood sugar levels, temperature, lactose levels, etc., treatment database 206 may include records indicative of characteristics of how the vital signs were obtained. For example, treatment database 206 may include data indicative of whether a particular vital sign measurement was taken invasively or non-invasively (the latter indicating a higher degree of clinician concern), how often a particular vital sign was taken/measured, a stated reason for taking the measurement, and so forth. More generally, treatment database 206 may include records indicative of characteristics of treatment provided to patients. These records may include but are not limited to whether a particular medicine or therapy was prescribed and/or administered, a frequency at which the medicine/treatment is prescribed/administered, an amount (or dosage) of medicine/treatment prescribed/administered, whether certain therapeutic and/or prophylactic steps are taken, whether, how frequently, and/or how much fluids are being administered, and so forth.

In some embodiments, machine learning classifier 216 may be trained using one or more patient feature vectors containing health indicator features obtained from health indicator database 204 and/or one or more treatment features obtained from treatment database 206. Once machine learning classifier 216 is sufficiently trained, it may receive, as input, patient feature vectors associated with subsequent patients, and may provide, as output, indications of levels of clinician acuity assessment pertaining to those subsequent patients. In essence, machine learning classifier 216 “learns” how previous patients were treated in response to a variety of health indicators, and then uses that knowledge to “guess” or “estimate” how one or more clinicians currently assess a patient's acuity based on a variety of the same signals. This guess or estimate, which as noted above may be referred to as the “CAAI,” may then be used for a variety of purposes.

One purpose for which a CAAI may be used is to assess a current patient's acuity. Medical assessment engine 208 may be accessible by one or more client devices 212 that may be operated by one or more medical personnel to determine a patient's acuity. In some embodiments, medical assessment engine 208 may classify a patient as having a particular level of acuity based on the CAAI of that patient. For example, the patient feature vector(s) may be provided as input to machine learning classifier 216, which in turn may provide a CAAI. The CAAI may then be returned to medical assessment engine 208, which may use the CAAI alone or in combination with other data points to provide an assessment of the patient's acuity. This assessment may be made available to medical personal at client devices 212, so that they can react accordingly. For example, suppose a new ER doctor is just beginning a shift. To quickly bring the ER doctor up to speed about multiple ER patients with which the doctor may not be familiar, the doctor may be provided (e.g., at any of client devices 212) with CAAI indicators for the patients, so that the doctor will quickly be able to ascertain which patients warrant the most urgent attention.

In some embodiments, medical assessment engine 208 or another component depicted in FIG. 2 may be configured to determine whether a current clinician assessment of the given patient's acuity is accurate based on the CAAI. For instance, medical assessment engine 208 may determine that the CAAI output by machine learning classifier 216 fails to satisfy a clinician acuity assessment threshold. In some embodiments, machine learning classifier 216 may be configured to map input vectors to output classes corresponding to “grades” or “scores” of clinician acuity assessment. If medical assessment engine 208 receives an indication from clinician acuity assessment determination engine 202 that machine learning classifier 216 has given the clinician acuity assessment a failing grade, medical assessment engine 208 may provide audio, visual, and/or haptic output, and/or cause such output to be provided on one or more client devices 212, to notify medical personnel that the current clinician assessment of the patient's acuity should be reevaluated.

Additionally or alternatively, in some embodiments, medical assessment engine 208 may be configured to determine whether an “objective” acuity level of the given patient matches (e.g., is within a predetermined range of) a CAAI estimated for the given patient based on health indicator and treatment features associated with the patient. In response, medical assessment engine 208 may cause output to be provided to medical personnel (e.g., at client devices 212) to instruct the medical personnel that a current clinician assessment of the patient's acuity is inaccurate. For example, the medical assessment engine 208 may choose to more actively output (e.g., with large or flashing text, alarm sounds, messages pushed to devices of the medical staff) the objective patient acuity measure.

As used herein, “objective” patient acuity may refer to an objective measurement (e.g., as output by a CDS algorithm) of the patient's acuity based solely on observable health indicators (e.g., age, pulse, blood pressure, gender, etc.), as opposed to the CAAI, which reflects clinician assessment of acuity, and is also based on characteristics of subjective treatment provided to the patient. Some example “objective” indices that may be used include the hemodynamic instability index (“HII”) or the early deterioration index (“EDI”), both developed by Philips Healthcare. Other “objective” indices may be calculated based on patient health indicators using various algorithms, such as algorithms for detecting acute lung injury (“ALI”) and/or acute respiratory distress syndrome (“ARDS”), to name a few. In various embodiments, multiple CAAI algorithms may be trained and deployed for pairing with one or more of these objective patient acuity measures. For example, a CAM for hemodynamic instability may be used for comparing clinician assessment to the HII, while a separate CAAI for EDI may be used for comparing clinician assessment to the EDI. In some embodiments, the output of a CAAI may be of the same type as output by the corresponding objective CDS algorithm such that the values can be directly compared. For example, where an objective CDS algorithm outputs a value on a scale of 1-to-10, the corresponding CAAI algorithm may also output a value on a scale of 1-to-10. As another embodiments, where an objective CDS algorithm outputs a classification, the corresponding CAAI algorithm may also output a classification.

In some embodiments, a manner in which an indicator of an objective acuity level of the given patient is output to medical personnel may be altered, e.g., by medical assessment engine 208, based on a comparison of an objective acuity level of a patient generated using one or more of the health indicator-based indices described above and a CAAI associated with the patient. Suppose medical assessment engine 208 determines that the CAAI of a patient “matches” (e.g., is within a predetermined range of) an objective acuity of the patient calculated using, say, the HIT. In such a scenario, medical assessment engine 208 may determine that clinicians are sufficiently concerned for the patient. Consequently, medical assessment engine 208 may cause one or more HII indicators that are output to medical personnel (e.g., displayed on a screen of one or more client devices 212) to be output less conspicuously, and/or not output at all, to avoid annoying or otherwise inundating medical personnel with too much information.

On the other hand, if medical assessment engine 208 determines that the CAAI of the patient does not match the patient's HII (or another similar objective acuity index), then it may be the case that medical personnel have underestimated a patient's deterioration. Accordingly, medical assessment engine 208 may cause one or more HII indicators to be output (e.g., on one or more client devices 212) more conspicuously, more often, etc., to put the medical personnel on notice of this discrepancy.

Medical assessment engine 208 or another component may make other decisions based on a CAM output by machine learning classifier 216 as well. In some embodiments, an ADT decision for patient may be may made based at least in part on a CAAI associated with the patient. As noted above, the CAAI can itself be used as a measure of patient acuity (in addition to its role as an indicator of clinician acuity assessment), and thus could dictate whether an amount of care required by a patient is low enough to justify discharging the patient and/or transferring the patient from an intensive care unit (“ICU”) to, for instance, a recovery unit. On the other hand, medical assessment engine 208 could determine, based at least in part on a patient's CAAI, that the patient should be transferred to an ICU from somewhere else, such as surgery or a triage station.

Yet another purpose for which a CAAI may be used is to adjust one or more medical alarms associated with one or more machines used to treat and/or monitor patients. In various embodiments, medical alarm engine 210 may be configured to select one or more thresholds or other criteria that, when satisfied, trigger one or more alarms. These thresholds and/or criteria may be made available to medical personnel (e.g., via client devices 212a-d) and/or at one or more medical machines (not depicted) configured to treat any or monitor patients.

Suppose a CAAI provided by machine learning classifier 216 is used to select a threshold associated with a vital sign or a combination of vital signs (e.g., min/max acceptable blood pressure, min/max acceptable glucose levels, min/max acceptable blood pressure/heart rate, etc.). Then, suppose that over time, medical understanding evolves or hospital best practices change, and that as a consequence, different treatment regimens evolve for responding to the same set of symptoms. Such evolution of medical treatment may cause a corresponding evolution of the CAAI, which in turn may lead to alteration of one or more medical alarms.

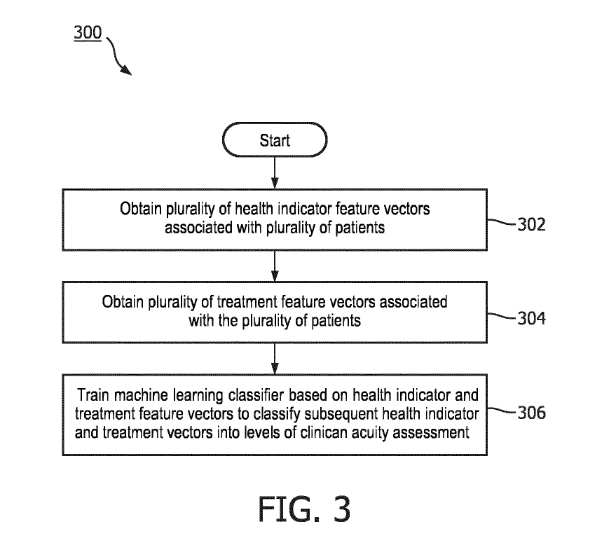

Referring now to FIG. 3, an example method 300 of training a machine learning classifier (e.g., 216 in FIG. 2) is depicted. For the sakes of brevity and clarity, the operations of FIG. 3 and other flowcharts disclosed herein will be described as being performed by a system. However, it should be understood that one or more operations may be performed by different components of the same or different systems. For example, many of the operations may be performed by clinician acuity assessment determination engine 202, e.g., in cooperation with machine learning classifier 216.

At block 302, the system may obtain a plurality of health indicator feature vectors associated with a plurality of patients, e.g., from health indicator database 204 in FIG. 2. As noted above, these health indicator feature vectors may include, as features, a wide variety of observable health indicators associated with patients. These health indicator features may include but are not limited to age, gender, weight, blood pressure, temperature, pulse, central venous pressure (“CVP”), electrocardiogram (“EKG”) readings, oxygen levels, genetic indicators such as hereditary and/or racial indicators, and so forth.

At block 304, the system may obtain a plurality of treatment feature vectors associated with the plurality of patients, e.g., from treatment database 206 in FIG. 2. Each treatment feature vector may include a plurality of treatment features associated with treatment of a given patient of the plurality of patients by medical personnel. In many instances, the treatments provided to the given patient may be based at least in part on (e.g., responsive to) a corresponding plurality of health indicator features of a health indicator feature vector associated with the given patient. A “treatment” may include any action taken by medical personnel on a patient's behalf, e.g., to administer drugs or therapy to the patient, or monitor one or more aspects of the patient, etc. A “treatment vector” may include one or more attributes or characteristics of one or more treatments provided by medical personnel to a patient. For example, a treatment may be to take a patient's blood pressure. A characteristic of taking a patient's blood pressure may be whether the blood pressure was taken invasively or non-invasively, how often the blood pressure is taken, and so forth. Similar characteristics may be associated with taking other health indicator measurements. As one non-limiting example, whether a Glasgow Coma Score (“GCS”) of a patient is measured, and how frequently it is measured, may be features of a treatment vector.

As another non-limiting example, a treatment vector may include a feature indicative of whether a patient is supported by a life-critical system such as a ventilator, a dialysis machine, and so forth. Additionally or alternatively, various operational parameters of life-critical systems used to treat/maintain/monitor a given patient may also constitute features of treatment vectors, such as whether the patient is on an arterial or venous line. As another non-limiting example, a treatment vector may include a feature indicative of a dosage, frequency, and/or duration of a medication or therapy administered to a patient. As another non-limiting example, a treatment vector may include a feature indicative of whether one or more labs have been ordered for a patient, such as whether lactate has been measured.

At block 306, the system may train a machine learning classifier (e.g., 216) based on the plurality of health indicator vectors obtained at block 302 and the corresponding treatment vectors obtained at block 304. In various embodiments, the machine learning classifier may be trained at block 306 to receive, as input, subsequent health indicator and treatment feature vectors, and to provide, as output, indications of levels of clinician acuity assessment (i.e. CAAI). As mentioned previously, in various embodiments, rather than being in two different vectors, health indicator features and treatment features may be incorporated into a single vector, or may be incorporated into more than two different vectors per patient.

The machine learning classifier may be trained in various ways. In some embodiments that employ supervised machine learning (e.g., using gradient descent), the machine learning classifier may be trained with a plurality of training examples. Each training example may consist of a pair that includes, as input, a health indicator and treatment vector (as two separate vectors or a single patient feature vector), and as desired output (also referred to as a “supervisory signal”), a “label.”

Various types of labels may be employed. In some embodiments, labels associated with patient outcome may be employed. Patient outcome labels may take various forms, such as positive, neutral, or negative, or various intermediate ratings. Additionally or alternatively, patient outcome labels may be indicative of various measures of acuity, such as mortality, morbidity, quality of life, length of stay (e.g., at hospital), amount of follow-up treatment required, and so forth. If multiple outcome metrics are employed, they may be weighted in various ways, depending on priorities, policies, etc. In some embodiments, a panel of clinicians may provide a weighting. They may agree to multiple measures of good or bad outcomes, e.g., death, severely impaired brain function, immobilization, etc. One possible approach is to use a small number of especially bad outcomes to label patients for a particularly undesirable acuity class, and to exclude “milder” but still negative outcomes from a more desirable class when training the classifier. Then, the classifier may be operated using the milder outcomes. The results of the classifier could be shown to the panel of clinicians to see whether it conforms with their intuitions. This may be iterated with negative outcomes of varying severity being used as negative labels in the training set, until the clinicians' intuitions are satisfied.

In various embodiments, a classifier may be trained to output CAAIs for different types of problems. For example, one machine learning classifier may be trained to output a CAAI for hemodynamic instability to be used with HII. Another machine learning classifier may be trained for AKI to be used with an index for AKI, etc. In some embodiments, patients who are designated DNR (do not resuscitate) or some similar designation (e.g., comfort measures only) may be excluded from training a machine learning classifier, because they may reject treatment in spite of having high acuity.

Based on these training examples, an inferred function may be produced that can be used to map subsequent health indicator/treatment vectors to likely patient outcomes. If a new health indicator/treatment vector associated with a new patient maps to a negative outcome, a determination may be made, for instance, that clinician assessment of the patient's acuity is inaccurate, and that the patient may warrant more medical care than is currently being provided and/or contemplated. Additionally or alternatively, in some embodiments, gradient descent or the normal equation method may be employed to train the machine learning classifier such as, for example, in the case where the machine learning classifier is represented as a logistic regression model or neural network model. Gradient descent or the normal equation method may also be used for other machine learning models such as, for example, linear regression models. As will be appreciated, various approaches to implementing gradient descent are possible such as for example, stochastic gradient descent and batch gradient descent.

In some embodiments, a machine learning classifier may be initiated, e.g., at a location such as a hospital or throughout a geographic area containing multiple medical facilities, e.g., in a preconfigured state (e.g., already trained with default training data). After initiation, a sliding temporal window (e.g., six months) of retrospective data may be used to update the machine learning classifier to recent and/or local best practices as they evolve.

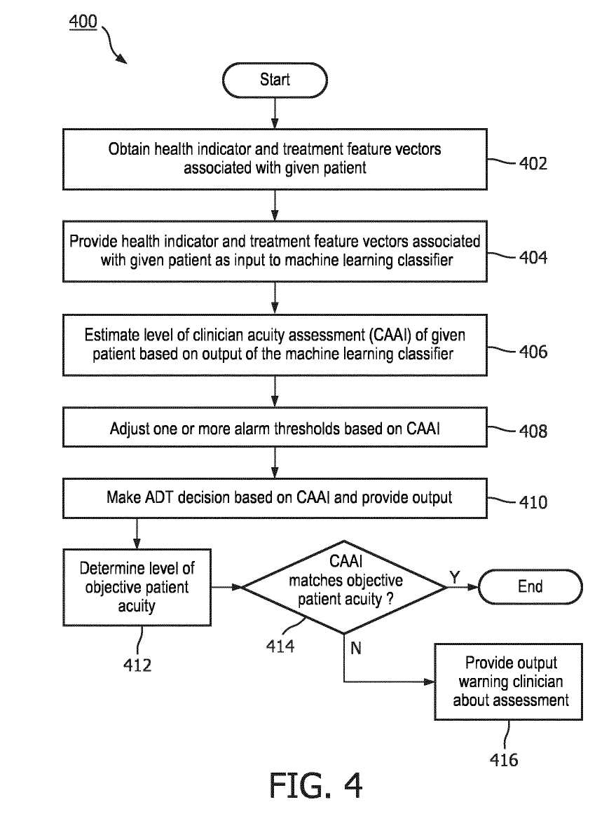

FIG. 4 schematically illustrates an example method 400 of using output of a machine learning classifier (e.g., CAAI) as 216 for various purposes. At block 402, health indicator and treatment vectors (which as noted above may be combined into one or more patient feature vectors) associated with a patient-of-interest may be obtained, e.g., from health indicator database 204 and/or treatment database 206 in FIG. 2. At block 404, the health indicator and treatment vectors obtained at block 402 may be provided as input to a machine learning classifier (e.g., 216 in FIG. 2). At block 406, a level of clinician acuity assessment (i.e. CAAI) of the patient-of-interest may be estimated based at least in part on output of the machine learning classifier.

The remaining operations of method 400 are optional applications of the CAM determined at block 406. For example, at block 408, one or more alarm thresholds maintained by, for instance, medical alarm engine 210 in FIG. 2, may be adjusted based at least in part on the estimated CAAI. In some embodiments, a CAAI may be used to evaluate an existing medical alarm. Suppose a CAAI indicates relatively low clinician concern, even in spite of one or more medical alarms being triggered. This may suggest that clinicians are ignoring the alarm (e.g., because they don't consider it serious or even false), and/or that the alarm is overused. Consequently, in various embodiments, medical alarm engine 210 may adjust the alarm to be less frequent, so that it is more likely to impact clinician concern.

At block 410, one or more ADT decisions may be made, and output may be provided as a result, based at least in part on the CAAI. For example, if the CAAI is relatively low, and there is no reason to question whether it shouldn't be higher, then medical personnel may be provided with output advising them to consider discharge of the patient and/or transfer to a lower-intensity medical treatment facility. At block 412, an objective acuity of the patient-of-interest may be determined using one or more of the techniques described above (e.g., HII, EDI, etc.), e.g., based on one or more features of the health indicator vector (but not the treatment vector) obtained at block 402. At block 414, the objective acuity of the patient-of-interest may be compared to the CAAI determined at block 406 to determine whether they “match.” As noted above, in some embodiments, an actual patient acuity and a CAAI associated with a patient “match” when they are within a predetermined range of each other. In some embodiments, one or both values may be normalized to aid in comparison.

If the answer at block 414 is no, then method 400 may proceed to block 416. At block 416, one or more health personnel may be provided with audio, visual, and/or haptic output, e.g., at one or more client devices 212, that indicate that the CAAI is likely incommensurate with the patient's actual acuity. In some instances, the clinician's assessment of the patient's acuity may underestimate the patient's actual acuity, in which case the clinician may be prompted to raise his or her level of concern. In other instances, the clinician's assessment of the patient's acuity may overestimate the patient's objective acuity, in which case the clinician may be prompted to reduce treatment and/or concentrate on other, higher acuity patients. If the answer at block 414 is yes, then method 400 may end.

One non-limiting technical advantage of training and using machine learning classifiers as described herein to estimate CAAI is that the machine learning classifiers can “tailor” themselves to reflect differences between medical knowledge and practices across spatial regions and/or across time, as well as across different practitioners and/or practices. For example, and as alluded to above, a machine learning classifier may evolve over time, e.g., as new medical knowledge leads to changes in standards of care and/or best practices. In addition, machine learning classifiers used in different geographic areas may operate differendy from each other due to a variety of factors, such as differences in standard of care and/or best practices between the geographical areas. Moreover, machine learning classifiers used by different practice groups and/or practitioners may operate differendy from each other due to a variety of factors, such as differences in standard of care and/or best practices between the practices/practitioners.

In some embodiments, a CAM may be used to develop new acuity indicators/indices and/or to refine existing indicators/indices. For example, a CAAI could be included as a feature in a patient episode vector that labels the episode as, for instance, high versus low clinical concern. Such patient episode vectors could then be used to train a machine learning classifier to better predict future high-clinical-concern episodes before they happen.

CAAIs may also be used to determine whether clinician concern is sufficient or insufficient over time, as well as to evaluate clinician consistency. For example, an expected CAAI for a given patient may be determined, e.g., based on similar historical instances known to yield positive outcomes. Then, an instant CAAI may be calculated for the patient and compared to the expected CAAI. If multiple instant CAAIs are lower than multiple expected CAAIs during a time period (e.g., during the night shift, between shifts, weekends, etc.), that may evidence insufficient monitoring. On the other hand, if multiple instant CAAIs are greater than multiple expected CAAIs during a time period, that may evidence excessive monitoring, in which case weaning of one or more therapies may be suggested. Additionally, one group of CAAIs (e.g., estimated during one time period, or from patients treated by a first medical team) could be compared to another group of CAAIs (e.g., estimated during another time period, or from patients treated by a second medical team) to determine how consistent clinician acuity assessment is between the two groups. Lack of consistency may suggest insufficient protocols, or insufficient compliance with protocols.

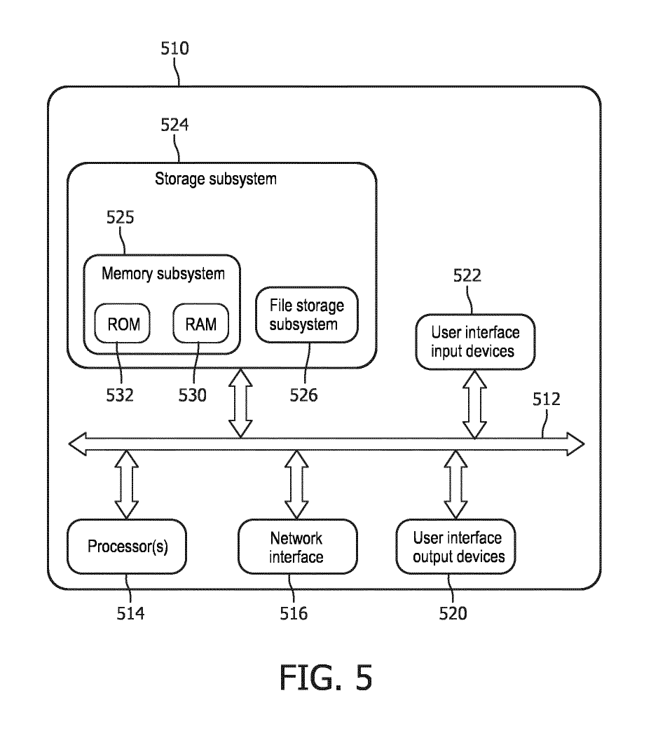

FIG. 5 is a block diagram of an example computer system 510. Computer system 510 typically includes at least one processor 514 which communicates with a number of peripheral devices via bus subsystem 512. These peripheral devices may include a storage subsystem 524, including, for example, a memory subsystem 525 and a file storage subsystem 526, user interface output devices 520, user interface input devices 522, and a network interface subsystem 516. The input and output devices allow user interaction with computer system 510. Network interface subsystem 516 provides an interface to outside networks and is coupled to corresponding interface devices in other computer systems.

User interface input devices 522 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computer system 510 or onto a communication network.

User interface output devices 520 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computer system 510 to the user or to another machine or computer system.

Storage subsystem 524 stores programming and data constructs that provide the functionality of some or all of the modules described herein. For example, the storage subsystem 524 may include the logic to perform selected aspects of methods 300 and/or 400, and/or to implement one or more of clinician acuity assessment determination engine 202, machine learning classifier 216, medical assessment engine 208, and/or medical alarm engine 210.

These software modules are generally executed by processor 514 alone or in combination with other processors. Memory 525 used in the storage subsystem can include a number of memories including a main random access memory (RAM) 530 for storage of instructions and data during program execution and a read only memory (ROM) 532 in which fixed instructions are stored. A file storage subsystem 526 can provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem 526 in the storage subsystem 524, or in other machines accessible by the processor(s) 514. As used herein, the term “non-transitory computer-readable medium” will be understood to encompass both transitory memory (e.g. DRAM and SRAM) and non-transitory memory (e.g. flash memory, magnetic storage, and optical storage) but to exclude transitory signals.

Bus subsystem 512 provides a mechanism for letting the various components and subsystems of computer system 510 communicate with each other as intended. Although bus subsystem 512 is shown schematically as a single bus, alternative implementations of the bus subsystem may use multiple busses.

Computer system 510 can be of varying types including a workstation, server, computing cluster, blade server, server farm, or any other data processing system or computing device. Due to the ever-changing nature of computers and networks, the description of computer system 510 depicted in FIG. 5 is intended only as a specific example for purposes of illustrating some implementations. Many other configurations of computer system 510 are possible having more or fewer components than the computer system depicted in FIG. 5.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of,” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope

1. A system comprising:
one or more processors; and
memory coupled with the one or more processors, the memory storing instructions that, in response to execution of the instructions by the one or more processors, cause the one or more processors to:
obtain a plurality of patient feature vectors associated with a plurality of patients, each patient feature vector including a plurality of health indicator features associated with a patient of the plurality of patients, and a plurality of treatment features associated with treatment of the patient by medical personnel based at least in part on the plurality of health indicator features associated with the patient; and
train a machine learning model based on the patient feature vectors including the plurality of treatment features associated with treatment of the patient by medical personnel to receive, as input, subsequent patient feature vectors, and to provide, as output, indications of levels of clinician acuity assessment;
provide one or more feature vectors that include health indicator features and
treatment features associated with a given patient to the machine learning model as input;
estimate a level of clinician acuity assessment of the given patient based on output of the machine learning model; and
performing at least one of:
adjusting one or more medical alarm thresholds based at least in part on the estimated level of clinician acuity assessment associated with the given patient; and
providing output to medical personal advising on whether to admit, discharge, or transfer the given patient based at least in part on the estimated level of clinician acuity assessment associate with the given patient.

2. The system of claim 1, wherein the memory further comprises instructions to:
adjust one or more medical alarm thresholds based at least in part on the estimated level of clinician acuity assessment associated with the given patient.

3. The system of claim 1, further comprising instructions to:
determine that the estimated level of clinician acuity assessment of the given patient fails to satisfy a clinician acuity assessment threshold; and
cause output to be provided to medical personnel to instruct the medical personnel that a current clinician assessment of the given patient's acuity is inaccurate.

4. The system of claim 1, further comprising instructions to determine that an objective acuity level of the given patient does not match the level of clinician acuity assessment of the given patient.

5. The system of claim 4, further comprising instructions to cause output to be provided to medical personnel to instruct the medical personnel that a current clinician assessment of the patient's acuity is inaccurate.

6. The system of claim 4, further comprising instructions to alter a manner in which an indicator of an objective acuity level of the given patient is output to medical personnel to notify the medical personnel that additional concern for the given patient is warranted.

7. The system of claim 1, wherein at least one patient feature vector includes at least one of:
a feature indicative of whether a health parameter of a patient is being measured invasively or non-invasively;
a feature indicative of a frequency at which a health indicator of a patient is measured;
a feature indicative of whether a patient is supported by a life-critical system; and
a feature indicative of a dosage or duration of a medication administered to a patient.

8. (canceled)

9. (canceled)

10. (canceled)

11. The system of claim 1, wherein each of the plurality of patient feature vectors includes a label indicative of an outcome associated with the respective patient.

12. A computer-implemented method, comprising:
obtaining, by one or more processors, a patient feature vector associated with a given patient, the patient feature vector including one or more health indicator features indicative of one or more observable health indicators of the given patient, and one or more treatment features indicative of one or more characteristics of treatment provided to the given patient;
providing, by the one or more processors, as input to a machine learning model operated by the one or more processors, the patient feature vector; and
estimating, by the one or more processors, based on output from the machine learning model, a level of clinician acuity assessment associated with the given patient.

13. The computer-implemented method of claim 12, further comprising adjusting one or more medical alarm thresholds based at least in part on the estimated level of clinician acuity assessment associated with the given patient.

14. The computer-implemented method of claim 12, further comprising providing output to medical personal advising on whether to admit, discharge, or transfer the given patient based at least in part on the estimated level of clinician acuity assessment associate with the given patient.

15. (canceled)

16. (canceled)

17. (canceled)

18. The computer-implemented method of claim 12 comprising:
determining by the one or more processors, based on the output from the machine learning model, a level of objective patient acuity measure;
comparing by the one or more processors, the objective acuity measure and the clinician acuity assessment for the given patient;
adjusting one or more medical alarm thresholds based at least in part on the estimated level of clinician acuity assessment associated with the given patient and the objective patient acuity measure.

19. (canceled)

20. (canceled)

21. (canceled)

22. (canceled)

23. (canceled)

24. The computer-implemented method of claim 12, further comprising: determining that an objective acuity level of the given patient does not match the level of clinician acuity assessment of the given patient.

25. The computer-implemented method of claim 12, further comprising: providing output to medical personnel to instruct the medical personnel that a current clinician assessment of the patient's acuity is inaccurate.

26. The computer-implemented method of claim 12, further comprising: altering a manner in which an indicator of an objective acuity level of the given patient is output to medical personnel to notify the medical personnel that additional concern for the given patient is warranted.
